(19) United States
(12) Patent Application Publication
(10) Pub. No.: US 2006/0135533 A1
(43) Pub. Date: Jun. 22, 2006

(54) Compound preparation for dizziness

(76) Inventor: Helga Schleenhain, Florsheim (DE)

Correspondence Address: KRIEGSMAN & KRIEGSMAN, 665 FRANKLIN STREET, FRAMINGHAM, MA, 01702, US

(21) Appl. No.: 10/542,279

(22) PCT Filed: Jan. 14, 2004
(86) PCT No.: PCT/DE04/00072

§ 371 Date: Jul. 15, 2005

(30) Foreign Application Priority Data
Jan. 15, 2003 (DE) 103 01 981.2

(51) Int. Cl. A61K 31/495

(52) U.S. Cl. 514/255.04

(57) ABSTRACT

The use of cinnarizine and dimenhydrinate or their physiologically compatible salts in combination is described for the treatment of vertigo of any genesis.

The present invention concerns the use of cinnarizine and dimenhydrinate or their physiologically compatible salts in combination.

Every tenth patient in general practice complains of vertigo. More than two million people annually visit their family physician due to disturbances in equilibrium. After headache, vertigo is thus the second most frequent symptom of disease.

Vertigo is frequently described by the patient as if he is experiencing rotational, swinging or lifting movements or the floor underfoot is unsteady. Others describe vertigo as a transitory loss of consciousness with confusion and insecurity of gait.

Three sensory systems are responsible for a person's orientation in space:

the optical system,

the vestibular system and

the proprioceptive system.

Vertigo is triggered by conflicting information from these three sensory systems due to lesions in peripheral or central equilibrium structures or due to ocular or psychogenic disturbances. Vertigo can also be an early sign of a serious disorder. Causes of vertigo can be vascular disorders, cardiac-circulatory disorders, metabolic disorders and disturbances in rheology. An interdisciplinary diagnosis is necessary due to this plurality of possible causes.

The classification of different forms of vestibular vertigo is usually conducted according to the site of emergence. One distinguishes between peripheral-vestibular, central-vestibular and combined central/peripheral-vestibular vertigo.

Information relating to the lesion site can be obtained from the manner in which the complaints of vertigo are manifested.

The differential diagnosis of vertigo is particularly supported by a comprehensive anamnesis. The anamnesis should contain questions relating to the type of vertigo, accompanying autonomic disturbances, vertigo-triggering situations or mechanisms, duration of attacks of vertigo and basic or accompanying disorders. A standardized anamnesis questionnaire, in which the course of the disease may also be documented, can be very helpful.

Tests for examining the vestibulo-oculary system are based on the fact that the equilibrium system responds to a labyrinth stimulus with reflex eye movements (nystagmus). Eye movements can be observed in patients directly by means of Frenzel glasses or recorded with the help of electronystagmography (ENG) or video-oculography (VOG). Parameters that can be evaluated include the number of nystagmus events per unit of time (nystagmus frequency), the velocity of the slow phase of nystagmus (GLP, also: slow phase velocity, SPV) as well as the nystagmus amplitude. Standard methods for stimulating the labyrinth include caloric testing with water or air, by means of which each labyrinth can be stimulated and examined individually, and the rotating chair test.

If nystagmus occurs even without stimulus (thus a so-called spontaneous nystagmus is present), diagnostic conclusions can be drawn from the direction of the nystagmus events.

For investigation of the vestibulo-spinal system, the Romberg standing test and the Unterberger step test are particularly suitable. The reactions of the patient can be observed directly and can be recorded by means of posturography or craniocorpography.

Different therapeutic approaches can lead to success, each time depending on the cause of the vertigo.

For medicinal therapy of vertigo, there are available, among others, antihistamines, parasympatholytics, cerebrally acting calcium antagonists, benzodiazepines, neuroleptics, medications that promote blood perfusion as well as homeopathics.

The selection of the optimal medicinal therapy is aligned with the cause of the vertigo.

The object of the present invention is thus to make available a therapeutic system which can provide therapy for all types of vertigo, i.e., vertigo of any genesis.

The object is solved by the use of cinnarizine and dimenhydrinate in combination.

One subject of the present invention is thus the use of cinnarizine and dimenhydrinate or their physiologically compatible salts in combination for the treatment of vertigo of any genesis.

Another subject of the present invention is the use of cinnarizine and dimenhydrinate or their physiologically compatible salts in combination for the preparation of drugs for the treatment of vertigo of any genesis.

The subject of the present invention is also the use of cinnarizine and dimenhydrinate or their physiologically compatible salts in combination along with pharmaceutically compatible adjuvants and/or additives for the preparation of drugs for the treatment of vertigo of any genesis.

The invention thus solves the problem of successfully treating all forms of vertigo—particularly the very frequently occurring forms of vertigo that cannot be clearly diagnosed—without erroneous therapeutical tests. Only a single medication is necessary due to the use according to the invention of the combination of cinnarizine and dimenhydrinate as active ingredients. This represents great progress in the therapy of vertigo.

The individual active ingredients that are used in combination according to the invention are known in and of themselves.

Cinnarizine (CAS 298-57-7) is the international nonproprietary name (INN) for 1-benzhydryl-4-trans-cinnamylpiperazine, [which] is an antihistamine and vasodilator and is described, for example, in U.S. Pat. No. 2,882,271.

Dimenhydrinate (CAS 523-87-5) is the international nonproprietary name (INN) for the 8-chlorotheophylline salt of diphenhydramine and is an antihistamine used as an antiemetic and against travel sickness, and is described, for example, in U.S. Pat. No. 2,499,058 or U.S. Pat. No. 2,534,813.

These active ingredients can be utilized according to the invention also in the form of their physiologically compatible salts. Common physiologically compatible inorganic and organic acids are, for example, hydrochloric acid, hydrobromic acid, phosphoric acid, sulfuric acid, oxalic acid, maleic acid, fumaric acid, lactic acid, tartaric acid, malic acid, citric acid, salicylic acid, adipic acid and benzoic acid. Other suitable acids, however, also include theophylline and its derivatives, such as, for example, 8-chlorotheophylline or other xanthines or caffeine and its derivatives. Other acids that can be used are described, for example, in Fortschritte der Arzneimittelforschung, Vol. 10, pages 224-225, Birkhäuser Publishers, Basel and Stuttgart, 1966, and Journal of Pharmaceutical Sciences, Vol. 66, pages 1-5 (1977).

The acid addition salts are usually obtained in a way known in and of itself by mixing the free base or its solutions with the corresponding acid or its solutions in an organic solvent, for example, a lower alcohol such as methanol, ethanol, n-propanol or isopropanol or a lower ketone such as acetone, methyl ethyl ketone or methyl isobutyl ketone or an ether such as diethyl ether, tetrahydrofuran or dioxane. Mixtures of the named solvents can also be used for better separation of crystals. In addition, physiologically compatible aqueous solutions of acid addition salts of the active ingredients used according to the invention can be prepared from an aqueous acid solution.

The acid addition salts of the active ingredients used according to the invention can be converted into the free bases in a way known in and of itself, e.g., with alkalis or ion exchangers. Other salts can be obtained from the free bases by reaction with inorganic or organic acids, particularly those which are suitable for the formation of salts for therapeutical use. These salts or also other salts of the new compound, such as, e.g., the picrate, can also serve for the purification of the free base by converting the free base into a salt, separating this salt and again releasing the base from the salt.

The subject of the present invention is also pharmaceuticals for oral, rectal, subcutaneneous, intravenous or intramuscular application, which contain a combination of the active ingredients according to the invention or their acid addition salt as the active ingredient, along with common vehicle and dilution agents.

The pharmacuticals of the invention are produced in the known way, with a suitable dosage, with the usual solid or liquid supports or dilution agents and the commonly used pharmaceutical technical adjuvants corresponding to the desired type of application. The preferred preparations consist of a form of administration which is suitable for oral application. Such administration forms are, for example, tablets, film tablets, dragees, capsules, pills, powders, solutions or suspensions or slow-release forms.

Of course, parenteral preparations such as injection solutions are also considered. In addition, suppositories, for example, can also be named as preparations.

Corresponding tablets can be obtained, for example, by mixing the active ingredient with known adjuvants, for example, inert dilution agents such as dextrose, sugar, sorbitol, mannitol, polyvinylpyrrolidone, bursting agents such as corn starch or alginic acid, binding agents such as starch or gelatins, slip agents such as magnesium stearate or talc and/or agents for achieving a slow-release effect, such as carboxypolymethylene, carboxymethylcellulose, cellulose acetate phthalate or polyvinyl acetate. The tablets can also consist of several layers.

Correspondingly, dragees can be produced by coating cores that have been produced analogously to the tablets with agents usually used in dragee coatings, for example, polyvinylpyrrolidone or shellac, gum arabic, talc, titanium dioxide or sugar. The dragee envelope may also consist of several layers, wherein the above-mentioned adjuvants for tablets can be used.

Solutions or suspensions containing the active ingredient according to the invention can also contain taste-improving agents such as saccharin, cyclamate or sugar, as well as, e.g., flavorings such as vanilla or orange extract. They may also contain suspension adjuvants such as sodium carboxymethylcellulose or preservatives such as p-hydroxybenzoate. Capsules containing active ingredients can be produced, for example, by mixing the active ingredient with an inert vehicle such as lactose or sorbitol and encapsulating it in gelatin capsules.

Suitable suppositories can be produced, for example, by mixing with support materials provided for this purpose such as neutral fats or polyethylene glycol or their derivatives.

The use of the combination of active ingredients according to the invention, among other things, demonstrates the following surprising properties:
The physician can successfully treat a far larger spectrum of vertigo patients with the combination of active ingredients used according to the invention than with the individual substances (expansion of therapeutic range, broadening of effective profile).
Cinnarizine as a single substance only has the indication “Vertigo for diagnostically clarified inner ear complaints, i.e., for peripheral vestibular complaints”.
Reduction of ingestion frequency
Simplification of the dosage plan
Improvement of compliance
It is observed surprisingly that with a dosage of the individual components that is reduced 2.5 times in the combination of active ingredients used according to the invention in comparison to the corresponding therapies with the individual substances, the effect was increased in a statistically significant manner with a demonstrated simultaneous reduction in the incidence of side effects.
Due to the application of the fixed combination in the combination of active ingredients used according to the invention, the synergistic, therapeutic effect can be optimally utilized.

The following examples explain the invention.

### EXAMPLES

Studies of effectiveness with the combination of active ingredients used according to the invention

The 12 clinical studies listed below were conducted; all of these were randomized and—with the exception of Experimental Study IX—were conducted double-blind according to the principles of “Good Clinical Practice”, i.e., according to the most recent international determinations.

Study I

This study involves a multicenter study, in which the combination of active ingredients used according to the invention was compared with the highest doses of the individual components that are commonly used in monotherapy: cinnarizine (50 mg) and dimenhydrinate (100 mg), as well as with placebo. The study centers were 3 ENT university clinics in Hungary (Budapest, Pecs, Debrecen). In all, 246 patients, who suffered from peripheral-vestibular, central-vestibular or the very frequently occurring combined form of peripheral-central-vestibular vertigo, were included in the study. As a result, the combination of active ingredients used according to the invention was demonstrated to be superior in a statistically highly significant manner (p≦0.001) to both the placebo as well as to the other therapies of highly dosed individual components

Study II

Patients with vertigo as a consequence of a diagnostically certain vertebro-basilar insufficiency were included in this placebo-controlled study at the ENT Clinic of the Medical Academy of Magdeburg. The combination of active ingredients used according to the invention was demonstrated to be statistically significantly superior to both betahistine (p≦0.01) as well as to placebo (p≦0.001).

Study III

Fifty patients with vertigo as a consequence of a vestibular neuropathy were included in this study at the Ear, Nose and Throat Clinic of the University of Rostock. Here, the combination of active ingredients used according to the invention was compared with the individual active ingredients cinnarizine (20 mg) and dimenhydrinate (40 mg)—i.e., with the same dosage as is present in the combination of active ingredients used according to the invention. As a result, the combination of active ingredients used according to the invention was shown to be statistically significantly superior to both individual components (p≦0.01).

Study IV

The combination of active ingredients used according to the invention was also compared with the individual components in “original concentration” (20 mg of cinnarizine or 40 mg of dimenhydrinate) in Study IV (3 centers: ENT Clinic of the University of Brünn, Sofia University Neurological Clinic, ENT Clinic of Pilsen). Patients who suffered from either central-vestibular, peripheral-vestibular, or combined peripheral-central-vestibular vertigo were included. The combination of active ingredients used according to the invention was shown to be statistically significantly superior to the individual components (p≦0.01) in this study also.

Study V

This 2-center study was conducted at the ENT clinics of the Medical Academy of Dresden and the Martin Luther University of Halle. Included were patients who suffered from peripheral, central, or combined peripheral-central vertigo. Comparative substances were the individual active ingredients at high dosage: cinnarizine (50 mg) and dimenhydrinate (100 mg). The combination of active ingredients used according to the invention was shown to be statistically highly significantly superior to the individual substances (p≦0.001).

Study VI

Study VI was conducted in the ENT Clinic, Pilsen. Included were patients with diagnostically certain inner ear vertigo. The comparative substance in this case was betahistine. As a result, a highly significant (p≦0.001) statistical superiority of the combination of active ingredients used according to the invention was shown when compared with the betahistine which is the standard treatment substance for this indication.

Study VII

This study was conducted in 3 study centers (ENT Clinics in Prague, Pilsen, Budweis). Here, the combination of active ingredients used according to the invention was tested against betahistine in patients with acute vertigo complaints. The combination of active ingredients used according to the invention was demonstrated to be statistically significantly superior to the betahistine (p≦0.05) in this study also.

Study VIII

The effect of the combination of active ingredients used according to the invention in comparison to betahistine was examined in study VIII in the ENT Clinic of the University of Olomouc in patients with diagnostically certain Meniere's disease. As a result, no statistically significant difference was shown between the combination of active ingredients used according to the invention and the betahistine of standard application in Meniere's disease.

Studies IX, X

Finally, 2 experimental studies were conducted with the combination of active ingredients used according to the invention against placebo or the combination of active ingredients used according to the invention against betahistine by the Aerospace Medicine Group of Johannes Gutenberg University in Mainz. Here, vertigo due to rotation with simultaneous execution of head movements was induced in healthy, volunteer subjects by means of an eccentric rotating chair—as also finds use in equilibrium training of astronauts, and the effect of the combination of active ingredients used according to the invention was compared with that of the placebo or with that of betahistine. In both studies, the effect of the combination of active ingredients used according to the invention was statistically significantly superior to the placebo (p≦0.01) or betahistine (p≦0.001) with respect to the number of tolerated head movements.

Studies XI, XII

Further, two studies were conducted for the determination of the influence of the combination of active ingredients used according to the invention on vigilance (ENT Clinic of the University of Würzburg; Institute for Brain Research, Bulgarian Academy of Sciences, Sofia). In both studies, the combination of active ingredients used according to the invention showed no statistically significant difference on vigilance both when compared to betahistine as well as when compared to placebo.

1. Use of cinnarizine and dimenhydrinate or their physiologically compatible salts in combination for the treatment of vertigo of any genesis.

2. Use of cinnarizine and dimenhydrinate or their physiologically compatible salts in combination for the production of pharmaceuticals for the treatment of vertigo of any genesis.

3. Use of cinnarizine and dimenhydrinate or their physiologically compatible salts in combination along with pharmaceutically compatible adjuvants and/or additives for the production of pharmaceuticals for the treatment of vertigo of any genesis.
